(19) United States
(12) Patent Application Publication
(10) Pub. No.: US 2005/0124852 A1
(43) Pub. Date: Jun. 9, 2005

(54) Method and device for the treatment of incontinence

(76) Inventors: Magnus Bolmsjo, Lund (SE); Sonny Schelin, Rockneby (SE)

Correspondence Address: JOHN R LEY, LCC, 5299 DTC BLVD, SUITE 610, GREENWOOD VILLAGE, CO, 80111, US

(21) Appl. No.: 10/928,743

(22) Filed: Aug. 27, 2004

(30) Foreign Application Priority Data
Aug. 29, 2003 (SE) 0302313-2

(51) Int. Cl. A61F 2/00; A61B 17/04

(52) U.S. Cl. 600/29

(57) ABSTRACT

A bulking agent is accurately administered into body tissue surrounding the urethra of a patient to support a urinary sphincter muscle and facilitate enhanced control over urination. Preferably, the bulking agent is inserted by use of a catheter which is anchored with a balloon in the bladder. The balloon contacts the bladder neck and locates the position of a needle which is extended from the catheter into the tissue. Consequently, the bulking agent is injected at a position relative to the bladder neck which is very effective in supporting the sphincter muscle. The catheter is rotated in rotational increments to introduce the bulking agent in multiple positions in an annular ring surrounding the urethra.

### TECHNICAL FIELD OF THE INVENTION

This invention concerns a method and device for the treatment of incontinence, and more particularly to delivering a bulking agent in a reliable position for achieving the best effect, preferably by using a treatment catheter comprising an expandable container attached at a front section for maintaining the treatment catheter in a fixed position for injecting the bulking agent.

### STATE OF THE ART

Urinary incontinence effects lifestyle in a dramatic manner. For many it means wearing damp undergarments, diapers, having skin irritation or diaper rash. It limits physical activity, social relationships, sexual attitudes, the ability to do spontaneous activities without careful planning, and in severe circumstances, may lead to a lonesome, hermit-like existence. Drug therapy, exercise programs, and biofeedback techniques have helped some patients, but a need for better treatment still remains. Stress incontinence due to a weakening of the pelvic floor muscles, often after childbirth, is one common cause of female incontinence.

Another treatment method is a technique called tension-free vaginal tape (TVT). The tape acts as a net around the neck of the bladder and supports it like a hammock. The tape is surgically inserted through the vagina to support the urethra, in a procedure that may last about 30 minutes. While the surgical procedure can be performed under general, regional, or local anesthesia, many surgeons prefer to use local anesthesia. After surgery, the tape will support the urethra during a sudden movement such as a cough or sneeze. The added support from the tape allows the urethra to remain closed and prevents the involuntary loss of urine. As is typically the case, the surgery may lead to problems and should be avoided for some categories of patients.

Treatment of urinary incontinence through endoscopic procedures using bulking agents has attained popularity in recent years. Dextranomer is effective as a nonimmunogenic bulking agent for the endoscopic treatment of urinary incontinence in the pediatric population, according to new research. Dextranomer is a synthetic co-polymer that has been used successfully for the treatment of vesico-ureteral reflux.

A 1% hyaluronan solution called DEFLUX has been used as a bulking agent for the treatment of urinary incontinence. Normally, an average of 2.5 cc of DEFLUX is endoscopically delivered to patients via a 4 F cannula at the bladder neck. For males delivery is made along the posterior urethra. DEFLUX has been used also for the treatment of vesicoureteral reflux (malformation of the urinary bladder) in children. Another medical implant, called NASHA (Non-Animal Stabilized Hyaluronic Acid) is used in a similar way.

Still another material is ZUIDEX which is intended for treatment of stress incontinence. ZUIDEX contains hyaluronic acid—a sugar molecule that is found in most tissues and bodily fluids where it provides structure and lubrication—and dextranomer—a sugar derivative that has been used for wound healing. It is injected into the urethra wall as a gel. An injecting device comprising four syringes and four extendable injection needles and a handle is used for injecting the gel in four different locations around the urethra.

A drawback with the injecting device is that it contains no means for facilitating the positioning of the injection needles. An exact positioning of the needles improves the possibilities of a successful treatment.

### THE INVENTION IN SUMMARY

It is an objective of the invention to overcome the above-mentioned drawback and to provide a device and a method that will ensure the reliable positioning and delivering of a bulking agent.

A device in accordance with the invention comprises a treatment catheter that is inserted through the urethra until a tip extends into the bladder. At the tip, the treatment catheter includes a balloon that can be inflated to a size sufficiently large to prevent it from being withdrawn from the bladder and moved through the urethra when in an inflated state. The treatment catheter has a length sufficient to reach the bladder from a position outside the body of the patient.

A hollow needle is provided in the catheter and can be extended radially from an opening or outlet in the catheter. The needle or an extension thereof, extends from a treatment position in the urethra to a position outside the body of the patient, where the bulking agent can be supplied to the needle. The needle is hollow to conduct the bulking agent to the tip, which is sharp to penetrate the tissue of the urethra. The needle has one or a plurality of openings for delivering the bulking agent or otherwise administering drugs.

In performing the treatment, the catheter is inserted in the urethra until the tip of the catheter is within the bladder, and the balloon is inflated. Then the catheter is retracted, to position the balloon against the bladder neck which surrounds the urethra where it opens into the bladder. As a result, the position of the catheter within the urethra and the outlet from which the needle is extended are well determined, preferably at a location along the length of the urethra from the bladder neck at which the injection of the bulking agent will have a desired effect. As a result, the needle can be extended at the intended position and the bulking agent can be delivered through the needle at the intended location.

The treatment catheter preferably possesses a high torsional stiffness to make it possible to rotate the catheter and insert the needle at different angles, all of which are located at the desired and intended position established by the balloon resting against the bladder neck.

The treatment catheter for female patients can be substantially shorter than the treatment catheter for male patients, due to the shorter urethra of women. The distance between the balloon and the needle outlet also varies depending on the sex of the patient.

### BRIEF DESCRIPTION OF THE DRAWINGS

The invention will now be described in more detail with the aid of exemplary embodiments and with reference to the accompanying drawings, in which

FIG. 1 is a side view partially in section of a first embodiment in accordance with the invention of a catheter for female patients;

FIG. 2 is a sectional view of an end of the catheter shown in FIG. 1 with connections for conducting liquid and the bulking agent within the catheter from outside of the patient;

FIG. 3 is a side view partially in section of a second embodiment in accordance with the invention of a catheter for male patients;

FIG. 4 is a side view of a female urethra and bladder after treatment in accordance with the invention;

FIG. 5 is a sectional view taken at line V-V shown in FIG. 1; and

FIG. 6 is longitudinal sectional view of a section of the treatment catheter shown in FIG. 1 adjacent to a tip end of the catheter.

### DETAILED DESCRIPTION

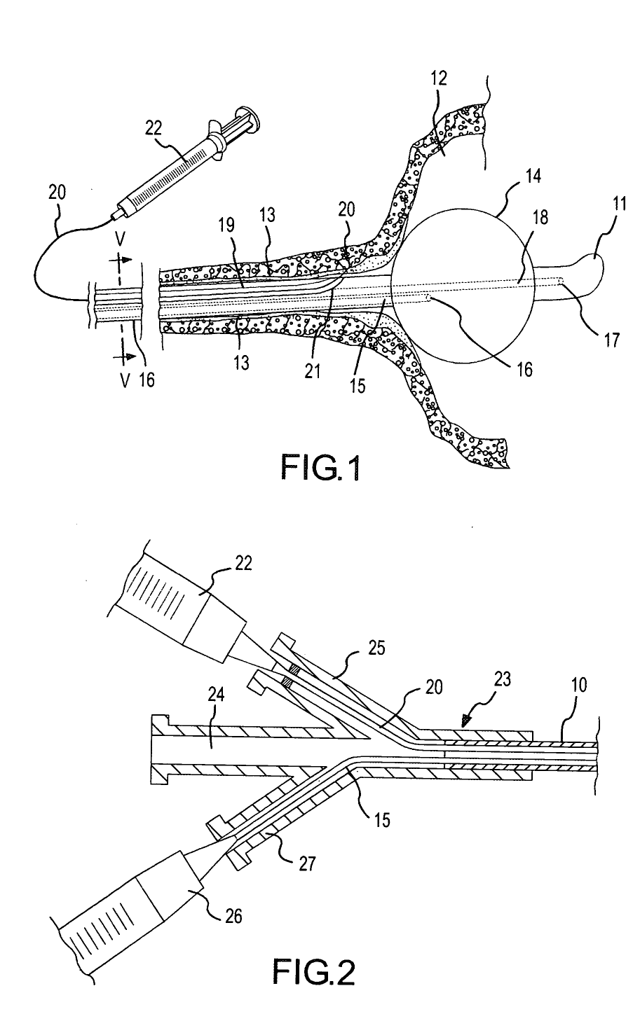

A treatment catheter 10 for women patients is shown in FIG. 1 in a treatment position within the urethra 13. A tip 11 at a first end of the catheter 10 extends into the bladder 12, and an opposite second end of the catheter is located outside the body of the patient (not shown). It should be noted that only a part of the bladder 12 is shown. An inflatable balloon 14 or anchor is close to the tip 11 on a section of the catheter 10 located within the bladder 12.

As shown in FIG. 1, the balloon 14 is inflated to a size sufficiently large to prevent unintentional withdrawal of the catheter 10 through the urethra 13. The size and location of the balloon 14 also ensures a predetermined axial or longitudinal position of the treatment catheter within the urethra 13, as will be further described below. Exerting a small pulling force on the catheter 10 from outside of the patient when the balloon 14 is inflated can further ensure the predetermined position of the catheter within the urethra. A first lumen 15 extends lengthwise through the catheter and has a first opening 16 into the balloon 14. The first lumen 15 is used to inflate the balloon with a suitable material, such as water, salt-solution or air.

An optional second opening 17 of an optional second lumen 18 is located at the tip. The second lumen 18 extends lengthwise inside the catheter 10 between the first and second ends of the catheter. The lumen 18 and its opening 17 can be used to drain urine from the bladder in connection with the treatment, or to otherwise communicate between the interior of the bladder and the second end of the catheter 10 outside of the patient.

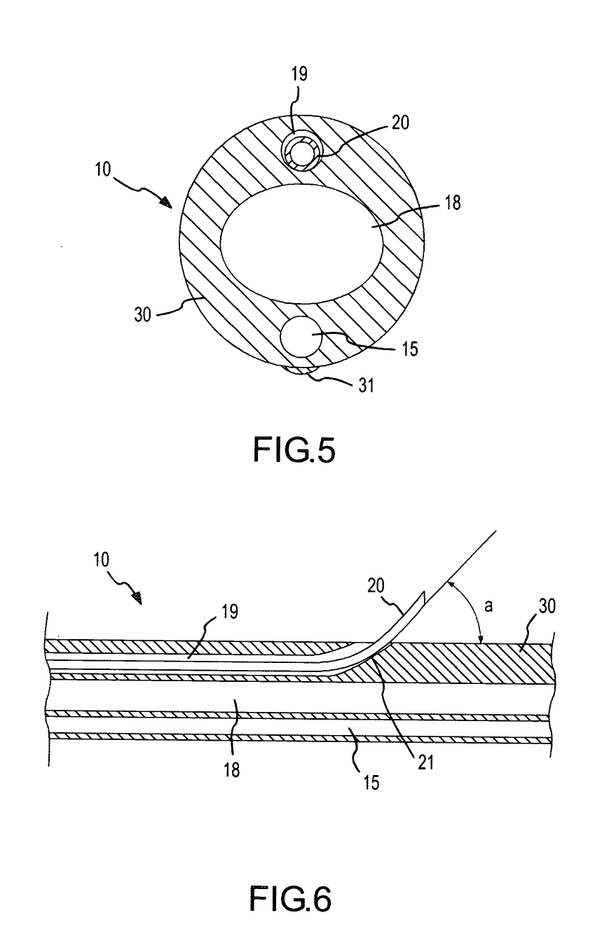

A third lumen 19 in the catheter extends from the second end of the catheter to an opening or outlet located at a predetermined distance from the inflatable balloon 14. At the outlet of the lumen 19, the outer wall of the catheter has a hole or a membrane or a section that can be penetrated by advancing a needle 20 through the lumen 19 and out of the outlet. The portion of the third lumen 19 adjacent to the outlet has a sloping section 21, shown in FIG. 6, which directs the needle 20 at a predetermined angle out of the catheter when a pushing force is exerted on the needle from outside of the patient at the second end of the catheter. The third lumen 19 constitutes a guide for the needle and can be replaced by or complemented with a tube having a bent end section at the outlet. The bent end or sloping section 21 is sufficiently strong to deflect the needle 20 into the urethra 13 at the desired angle upon advancement of the elongated needle out of the outlet of the lumen 19.

The needle 20 or an extension thereof extends from the second outside end of the catheter to the outlet of the lumen 19 which is spaced from the balloon 14. The needle 20 has a pointed end which is sufficiently sharp to penetrate the tissue surrounding the urethra 13. At the second outside end of the catheter 10, the needle 20 is connected to a supply device 22 for delivering the bulking agent or gel through the hollow needle. The supply device 22 can comprise a conventional syringe or any similar device. The needle 20 is arranged with its pointed end inside the catheter 10 and the lumen 19 during insertion of the catheter 10 in the urethra 13.

Shown schematically in FIG. 2 is a dual port Y-connector 23 used for connecting the bulking agent supply device 22 to the needle 20 and for connecting a similar supply device 26 for supplying an appropriate inflation fluid through the lumen 15 to the balloon 14 to the catheter 10. A central hollow space 24 in the Y-connector 23 can be used for draining urine from the bladder through the lumen 18. The bulking agent supply device 22 is connected to a first port 25 of the connector 23, and the inflation supply device 26, such as a syringe, is connected to a second port 27 of the connector 23. An outer or rear end of the needle 20, or an extension part thereof, is moved in and out of the first port 25 to extend and withdraw, respectively, the forward sharp end of the needle 20 from the opening position of the lumen 19 at the catheter wall.

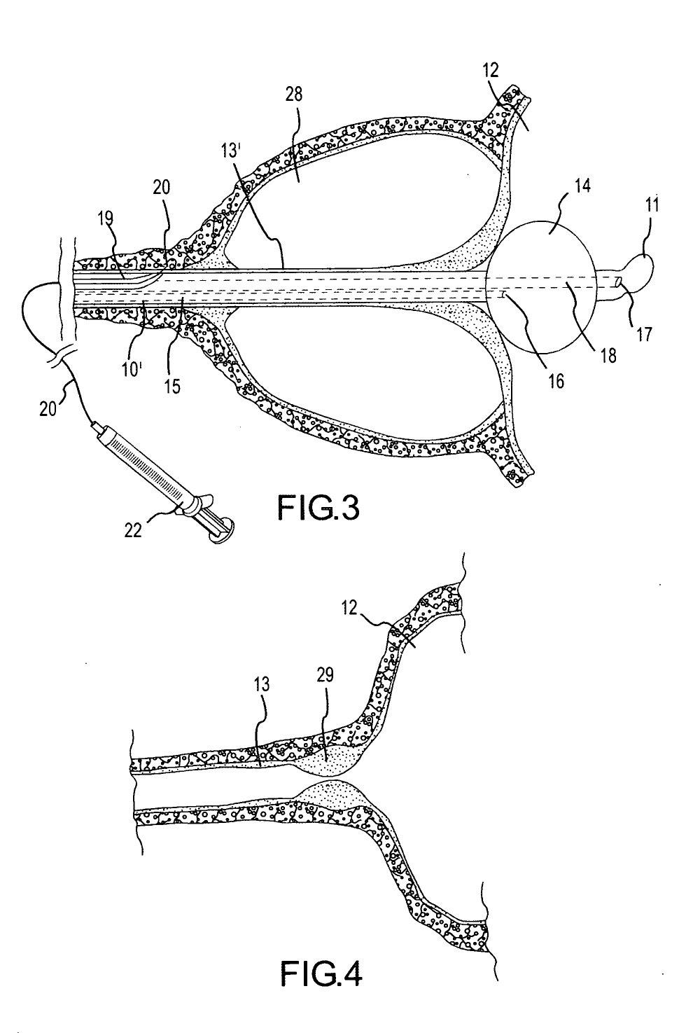

FIG. 3 shows a treatment catheter 10′ for male patients. The treatment catheter 10′ comprises the same elements as the female catheter 10 (FIG. 1). A tip 11 at the forward end of the catheter 10′ is formed to facilitate the insertion of the catheter 10′ through the male urethra 13′, which is substantially longer for male patients than the female urethra 13 (FIG. 1) is for female patients. The inflatable balloon 14 is provided near the tip 11 of the catheter 10′, and the first lumen 15 is arranged in connection with the balloon 14 for inflating it. The optional second lumen 18 with the optional second opening 17 at the tip of the catheter 10′ can be used for draining the bladder 12. In the third lumen 19, the needle 20 is moved axially or longitudinally, so as to be entirely within the lumen 19 of the catheter 10′ or extended outward from the outlet of the lumen 19 of the catheter 10′ into the tissue surrounding the urethra 13′ for injecting the bulking agent during the treatment process. The treatment catheter 10′ is flexible and pliable in order to be introduced flexibly through the urethra 13′ to the treatment position in a male patient.

A comparison of FIGS. 1 and 3 makes it apparent that the distance between the balloon 14 and the outlet of the lumen 19 where the needle 20 is extended from the catheter wall is larger in the catheter 10′ for male patients than in the catheter 10 for female patients. The reason for the difference in the distance between the balloon in the outlet of the lumen 19 is that for male patients a prostate gland 28 (FIG. 3) is located distally of the bladder 12 and the tissue for treatment in male patients is located distally of the prostate gland 28.

The relative terms “proximal” and “distal” are used in this description in relation to the patient and his or her urethra 13′ or 13, respectively. The portions of the urethra 13′ or 13 adjacent to the bladder 12 are more closely located to the central core of the patient and are therefore regarded as occupying “proximal” positions relative to the patient. The portions of the urethra 13′ and 13 which are located near the exterior opening of the urethra on the outside of the patient are more remotely located from the central core of the patient and are therefore regarded as occupying “distal” positions relative to the patient. Accordingly, the portions of the patient's anatomy which are the most internal within the patient are referred to as “proximal,” and the portions of the anatomy which are the most external of the patient are referred to as “distal.” The distal portions of the urethra are therefore downstream relative to the normal direction of urine flow through the urethra, and the proximal portions of the urethra are therefore upstream relative to the normal direction of urine flow through the urethra.

In a treatment process the catheter tip 11 is inserted in the urethra of the patient until the tip 11 reaches a location where it is positioned in the bladder 12. The balloon 14 is inflated within the bladder 12, and the bladder is optionally drained through the second lumen 18. By exerting a pulling force on the second end of the catheter when the balloon 12 is inflated, the inflated balloon 12 will contact the bladder neck and the catheter will be positioned in a predetermined axial or longitudinal location in the urethra relative to the bladder neck. Once in this position, the needle 20 is extended from the outlet of the lumen 19 by pushing the needle 20, or an extension thereof, at the second end of the catheter.

The sharpened forward tip end of the needle 20 will penetrate the mucous membrane of the urethra and the surrounding tissue, and the tip of the needle 20 will be positioned for delivering the bulking agent at a radial distance from the catheter. Normally, the radial distance is 3-10 mm, and normally approximately 10 mm. After administering the appropriate amount of the bulking agent at one injection position, the tip end of the needle 20 is pulled back into the lumen 19, and the complete catheter is rotated through an angular interval of approximately 90° around its longitudinal axis. The needle is extended into the tissue in a second position which is angularly rotationally displaced from the previous location where the bulking agent was injected. The bulking agent is then delivered at this second position which is rotationally angularly displaced from the previous location. The catheter should also have a torsional stiffness characteristic to assure that the rotational angle or interval for extending the needle can be directly controlled by rotating the second end of the catheter from outside the body.

Preferably, the steps of extending the needle, injecting the bulking agent, withdrawing the needle and rotating the catheter in another rotational interval are repeated, in two further steps (four total rotationally displaced injection positions) when the catheter is rotated 90° in each rotational interval between the adjacent angularly rotationally displaced injection portions, thus ending up with bulking agent injected in four different angularly displaced positions which circumscribe the urethra in an annular manner. Of course, the amount of the rotational interval may be adjusted as desired or necessary to inject the amount of bulking agent circumscribing the urethra as required to achieve an effective treatment for urinary incontinence. It is also possible to provide two diametrically opposite positioned lumens 19, each with its own needle 20, within a single catheter 10 or 10′, or a plurality of distributed lumens 19 for needles 20, and in so doing enable simultaneous injection of the bulking agent at two or more positions circumscribing the urethra. After completing the treatment, the first lumen 15 is used to empty the balloon 14 to allow the treatment catheter to be removed.

The balloon 14 fullfills an important function in regard to injecting the bulking agent. The inflated balloon 14 will locate or anchor the catheter in a predetermined location within the urethra by maintaining the first end of the catheter in a fixed position in the bladder as a result of the inflated balloon 14 contacting the bladder neck. By pulling the catheter at the second end, as will be done preferably in advance of each injection of the bulking agent, the catheter will be positioned in the urethra so that the balloon will contact the bladder neck. As a result and because the lumen 19 is retained in a fixed position within the catheter, the outlet of the lumen 19 from which the needle protrudes out of the catheter is also very well determined. Consequently, each separate injection of the bulking agent is located at approximately the same distance distal of the bladder neck. The outlet of the lumen 19 from which the needle protrudes out of the catheter is normally is 2-40 mm behind the balloon for female patients and is normally 5-100 mm behind the balloon for male patients. It is appropriate to provide different catheters with predetermined sizes and different needle extension positions to fit different physical conditions.

FIG. 4 illustrates the state of the tissue surrounding urethra after the bulking agent has been injected during the treatment. In four (two are shown) different locations, and in other locations when a rotational interval other than 90° is used, a circumferential sphere 29 of bulking agent has been placed distally of the bladder 12. The spheres 29 form an annular ring of bulking agent in the tissue which circumscribes the urethra at a position distal of a sphincter muscle at the bladder neck of a female patient (FIG. 1), and circumscribes the urethra at a position distal of an external sphincter muscle distal of the prostate gland 28 of a male patient (FIG. 3). Each separate injection of the bulking agent which forms a sphere 29, and all of the spheres 29 are located at approximately the same longitudinal location along the urethra, as a result of the positioning established by the balloon 14 contacting the bladder neck in the bladder 12. Locating the separate spheres 29 of injected bulking agent at approximately the same longitudinal location causes injections of bulking agent to cooperate better with one another in forming the annular ring of spheres 29, and a better formed annular ring of spheres 29 results in providing additional support and lift for the sphincter muscle to avoid and reduce urinary incontinence problems.

FIG. 5 illustrates, in a cross sectional view of one embodiment of a treatment catheter in accordance with the invention, how the lumens 15, 18 and 19 can be arranged in the catheter. The first lumen 15 extends in a wall 30 of the catheter and is used for inflating the balloon 14. The second lumen 18 runs axially along the catheter in a central position, circumscribed by the wall 30. When the catheter is in the treatment position, the second lumen 18 may be used to drain the bladder. Finally, the third lumen 19 extends in the wall 30 of the catheter opposite from the first lumen 15 and functions as a support or guide for the needle 20.

In the embodiment shown in FIG. 5, the exterior surface of the wall 30 of the catheter has a circular cross section while exterior surface of the second lumen 18 has an oval cross section. Other cross sections, such as an oval cross section of the catheter and a circular cross section of the second lumen, can be used. It is important that the rotational position of the catheter can be determined, because the catheter is rotated through different angular intervals into different positions during treatment, as described above. In the embodiment shown in FIG. 5, a projection or rib 31 is provided on the outer surface of the catheter along at least a section of the catheter for use in determining the rotational position of the catheter. It is possible also to use a color marking or a longitudinal indentation for this purpose. The needle 20 is also hollow, as shown in FIG. 5. The hollow interior opening of the needle conducts the bulking agent from the supply device 22 (FIG. 2) to and out of openings in the sharpened tip end of the needle 20.

FIG. 6 also illustrates an example of the location of the lumens 15, 18 and 19 within the catheter 10. The first lumen 15 runs along a bottom part (as shown) of the catheter and the second lumen 18 runs in a central part thereof. The dimensions of the lumens may vary. The third lumen 19 runs in parallel with the first lumen 15 and the second lumen 18, but the third lumen 19 ends at the forward outlet with an angled or sloping section 21. When the needle 20 is pushed inwards or forward in the third lumen 19, the forward portion of the needle 20 will be deflected at an angle “a” determined by the sloping section 21. The angle “a” can be from 10° to 90°. Preferably the angle “a” is approximately 30°.

In the manner described above, the injections of the bulking agent at reliably located positions circumscribing the urethra measured relative to the bladder neck results in the bulking agent creating a more complete and better positioned annular ring surrounding the urethra. The ability to inject the bulking agent at reliable and predetermined positions measured relative to the bladder neck is achieved by the use of the balloon on the catheter, and consistently positioning the balloon in contact with the bladder neck prior to each injection. Providing sufficient torsional stiffness of the catheter allows the catheter to be rotated at desired and controllable rotational intervals to distribute each of the bulking agent injections at desired and more precise circumferential positions surrounding the urethra, thereby establishing better effects created by the bulking agent in the continuous annular ring surrounding the urethra. The annular ring of bulking agent resulting from this treatment is better formed and is more effective in supporting the sphincter muscle, thereby enhancing the effectiveness of the sphincter muscle to reduce or eliminate urinary incontinence problems. Many other advantages and improvements will be apparent upon gaining a more complete appreciation for the invention.

Presently preferred embodiments of the invention and many of its improvements have been described with a degree of particularity. This description is of preferred examples of implementing the invention, and is not necessarily intended to limit the scope of the invention. The scope of the invention is defined by the following claims.

1. A device for treatment of urinary incontinence, comprising at least one hollow needle extendable from a support, wherein:
the support is a catheter having a first end with a tip and a second end with a connector;
the catheter includes an anchor in the vicinity of the tip;
the catheter supports the needle to allow it to move between a first position within the catheter and a second position extending out of the catheter at an outlet;
the needle includes at least one opening from the hollow at a tip of the needle;
the outlet is located at a predetermined distance from the inflatable balloon which is related to the distance between a bladder and a sphincter muscle surrounding a urethra of a patient upon which the treatment device will be used; and
the connector at the second end of the catheter comprises a first port for connecting a supply device to the hollow needle for supplying a bulking agent through the hollow needle to each opening from the hollow at the tip of the needle.

2. A device according to claim 1, wherein the tip of the needle is extendable from the catheter to a radial distance of approximately 3-10 mm from the catheter.

3. A device according to claim 1, wherein the needle is extendable from the catheter at an angle of 10°-90° relative to a longitudinal dimension of the catheter.

4. A device according to claim 1, wherein the needle is extendable from the catheter at an angle of approximately 30° relative to a longitudinal dimension of the catheter.

5. A device according to claim 1, wherein the catheter has sufficient rotational stiffness to rotate the catheter when positioned in the urethra of the patient through a predetermined rotational interval by rotational force applied to the second end of the catheter.

6. A device according to claim 1, wherein the anchor comprises an inflatable balloon.

7. A method for treatment of urinary incontinence in a patient having a urethra connected to a bladder at a bladder neck and also having a urinary sphincter muscle surrounding the urethra to control the flow of urine, including:
inserting a catheter through the urethra until a forward tip and an adjacent forward section of the catheter are located in the bladder;
inflating a balloon located on the catheter at the foreword section in the bladder to a size substantially larger than the urethra, to prevent unintentional retraction of the catheter in the urethra and to locate the catheter within the urethra relative to the bladder neck;
contacting the inflated balloon with the bladder neck;
extending a needle from the catheter into body tissue surrounding the urethra at a predetermined distance from the inflated balloon in the bladder neck related to the distance between the bladder and the sphincter muscle;
administering a bulking agent into the body tissue through the extended needle at the predetermined distance and at a position radially spaced from the urethra;
retracting the needle to a position within the catheter after administering the bulking agent through the extended needle;
rotating the catheter through an angular rotational interval; and
repeating the aforesaid contacting, extending, administering and retracting steps until the bulking agent is administered in a plurality of the positions radially spaced from the urethra which are distributed around the urethra in an annular ring located at the predetermined distance from the bladder neck.

8. A method of treating urinary incontinence by injecting a bulking agent in body tissue surrounding a urethra which extends from a bladder at a bladder neck where the urethra joins the bladder, comprising:
inserting a guide into the urethra;
locating the guide within the urethra at a predetermined location relative to the bladder neck;
extending a needle from the guide into the body tissue spaced from the urethra at a predetermined position relative to the bladder neck established by the predetermined location of the guide relative to the bladder neck; and
delivering the bulking agent from the extended needle into the body tissue at the predetermined position spaced from the urethra.

9. A method as defined in claim 8, further comprising:
extending the needle through the guide by force applied from outside of the urethra; and
introducing the bulking agent into the needle at the location outside of the urethra.

10. A method as defined in claim 9, wherein the interior of the needle is hollow, and the method further comprises:
flowing the introduced bulking agent through the hollow of the needle from the exterior of the urethra to the predetermined position where the bulking agent is delivered.

11. A method as defined in claim 8, further comprising:
rotating the guide within the urethra after delivering the bulking agent to the predetermined position;
locating the rotated guide within the urethra at the predetermined location relative to the bladder neck; and
delivering the bulking agent into the body tissue from the extended needle at another predetermined position which is angularly displaced from the predetermined position in which the bulking agent was previously delivered into the tissue spaced from urethra prior to rotating the guide.

12. A method as defined in claim 11, further comprising:
continuing the aforesaid steps of rotating the guide, locating the rotated guide at the predetermined position and delivering the bulking agent at another angularly displaced predetermined position until the bulking agent has been delivered in an annular ring surrounding the urethra at the predetermined location.

13. A method as defined in claim 12, further comprising:
retracting the extended needle from the tissue into the guide after delivering the bulking agent into the tissue at each predetermined position; and
rotating the guide within the urethra after the needle is retracted.

14. A method as defined in claim 11, further comprising:
rotating the guide by applying force to the guide from outside of the urethra.

15. A method as defined in claim 11, wherein the guide is located within a catheter and the catheter includes an anchor located at a forward end of the catheter, and the method further comprises:
inserting the catheter into the urethra until the forward end of the catheter is located in the bladder; and
contacting the anchor with the bladder neck to locate the catheter in the urethra relative to the bladder neck and thereby position the guide at the predetermined location relative to the bladder neck.

16. A method as defined in claim 15, further comprising:
rotating the guide by rotating the catheter within the urethra.

17. A method as defined in claim 16, further comprising:
rotating the catheter within the urethra by applying force to the catheter from outside of the urethra.

18. A method as defined in claim 17, further comprising:
transmitting the force applied from outside of the urethra throughout the catheter by utilizing a torsional resistance characteristic of the catheter.

19. A method as defined in claim 18, further comprising:
continuing the aforesaid steps of rotating the catheter and guide, locating the rotated catheter and guide at the predetermined position and delivering the bulking agent at another angularly displaced position until the bulking agent has been delivered in an annular ring surrounding the urethra at the predetermined position;
retracting the extended needle from the urethra into the guide after delivering the bulking agent into the urethra at each predetermined position; and
rotating the catheter and guide within the urethra after the needle is retracted.

20. A method as defined in claim 15, wherein the anchor comprises a balloon, and the method further comprises:
inflating the balloon within the bladder; and
contacting the balloon with the bladder neck to locate the catheter and guide in the urethra at a predetermined position relative to the bladder neck.
